(19) United States
(12) Patent Application Publication
(10) Pub. No.: US 2023/0310933 A1
(43) Pub. Date: Oct. 5, 2023

(54) HEARING DEVICE WITH HEALTH FUNCTION

(71) Applicant: Sonova AG, Stäfa (CH)

(72) Inventor: Hans-Ueli Roeck, Hombrechtikon (CH)

(21) Appl. No.: 17/657,373

(22) Filed: Mar. 31, 2022

(51) Int. Cl. A63B 24/00

(52) U.S. Cl. CPC A63B 24/0062; A63B 2024/0065; A63B 2024/0068

(57) ABSTRACT

A system includes a user-worn device such as a hearing device having a physiological sensor for measuring physiologic data or physical activity data of a user, and a user computing device. The user computing device is in communication with the user-worn device executes an app that obtains the physiologic data or physical activity data of the user from the user-worn device. The user computing device also, for each of a plurality time recurring time periods (e.g., one week), evaluates the obtained data for a current or prior time period and determines a current physical activity level based on the evaluated data, and adaptively determines a physical activity goal for a coming time period based on the determined current physical activity level.

### BACKGROUND

Wrist worn health devices and hearing devices are currently capable of measuring physical activities and physiological information. For example, these measurements may include step counts, duration of walking and running, weekly intensity minutes (e.g., as defined by World Health Organization guidelines as at least 150 minutes of moderate activity and/or 75 minutes of vigorous activity), and heart rate related measures (e.g., current heart rate, resting heart rate, stress levels, heart rate variability, oxygen saturation levels (SpO2), maximum tidal oxygen volume per minute under vigorous physical activity conditions (VO2max). Historical data of these measurements and statistical measures thereof can also be maintained. However, users of these devices are left alone with the measures. Without proper education or guidance, the user is not able to utilize the measures to monitor or improve their health or fitness.

Even where devices allow users to set goals to guide activity, such static goals are often far too ambitious or even dangerous for a sedentary or otherwise unfit person to start with. And still, the user is again left alone to set and adapt such goals to their current needs (e.g., to avoid no over-training and injury). For example, after a day of significant activity (e.g., 40,000 steps), a daily step goal may be overly increased (e.g., from 10,000 steps/day to 25,000 steps/day). However, in such circumstances a user should recover and, at least for a following day, reduce the number of steps. Moreover, such goal adjustments often are only based on activity levels alone, not corresponding physiological measures. As a result, where goals are adjusted, activities are often increased too fast and with negative effects. Such negative effects still further lead to de-motivation and a return to a sedentary life style.

For devices that provide suggested goals, those too suffer from the above-noted deficiencies. Further, such suggestions require an input of initial baseline data for the user. But for a currently inactive user, such information is not available and may even be de-motivating in the first place.

### BRIEF SUMMARY

According to one example of the present disclosure, a system comprises: a hearing device configured to be worn at an ear of a user, the hearing device comprising a microphone, a speaker, a processor, and at least one sensor configured to measure physiologic data or physical activity data of the user; and a user computing device configured to: obtain the physiologic data or physical activity data of the user from the hearing device, for each of a plurality of recurring time periods, evaluate the obtained physiologic data or physical activity data for a current or prior time period and determine a current physical activity level based on the evaluated data for the current or prior time period, and adaptively determine a physical activity goal for a coming time period based on the determined current physical activity level, and output the determined physical activity goal.

In various embodiments of the above example, the at least one sensor comprises a photoplethysmography sensor; the at least one sensor comprises an accelerometer; the recurring time period is one week; the determined physical activity goal for the coming time period is 50-80% of a physical activity goal of the prior time period when the current physical activity level is less than the determined physical activity level of two prior time periods earlier; the determined physical activity goal for the coming time period is less than, and at least 80% of, a physical activity goal of the prior time period when the coming time period is at a predetermined regular interval; the determined physical activity goal for the coming time period is greater than, and at most 110% of, a physical activity goal of the prior time period when the current physical activity level is greater than the determined physical activity level of two prior time periods earlier; the determined physical activity goal for the coming time period is the same as a physical activity goal for the prior time period when the current physical activity level meets or exceeds an established guideline activity goal; the determined physical activity goal for the coming time period is more than 100% and at most 110% of a physical activity goal for the prior time period when the current physical activity level meets or exceeds an established guideline activity goal; the determined physical activity goal for the coming time period is the same as a physical activity goal for the prior time period when the current physical activity level does not meet or exceed an established guideline activity goal; the determined physical activity goal for the coming time period is 110-120% of a physical activity goal for the prior time period when the current physical activity level does not meet or exceeds an established guideline activity goal; the user computing device is further configured to: transfer money from the user to a third party based on whether the determined current physical activity level meets or exceeds a physical activity goal for the prior time period; the user computing device is further configured to: transfer money from the user to a third party based on whether the determined current physical activity level does not meet or exceed a physical activity goal for the prior time period; the user computing device is further configured to: notify a third party of whether the determined current physical activity level meets or exceeds a physical activity goal for the prior time period; the user computing device is further configured to: notify a third party of whether the determined current physical activity level does not meet or exceed a physical activity goal for the prior time period; and/or the user computing device is further configured to: notify the user when the current physical activity level does not meet or exceed a physical activity goal of a current time period, prior to expiration of the current time period.

### BRIEF DESCRIPTION OF THE SEVERAL VIEWS OF THE DRAWING

FIG. 1 illustrates a system according to the present disclosure.

FIG. 2 illustrates an example flow chart for setting activity level goals according to the present disclosure.

FIG. 3 illustrates example activity level goals for actual activity levels determined by the system of the present disclosure.

FIG. 4 illustrates example incentive and motivation functionality of an app of a system of the present disclosure.

### DETAILED DESCRIPTION OF THE DRAWING

Based on the foregoing deficiencies, the present disclosure relates to a user-worn device with health functions that provides reasonable goals to users. More particularly, such goals may not be directed to static activity (e.g., running for a predetermined time or distance) and may not be based on a user's initial baseline activity level or ability. Further, such goals may be adaptive based on both a user's current activity level and/or ability, and physiological state (e.g., current fitness state, resting state, stress levels). Additionally, because physical activity is often not considered entertainment, and can be associated with discomfort and embarrassment, the present disclosure also relates providing the goals in a manner that is simple and motivating to the user.

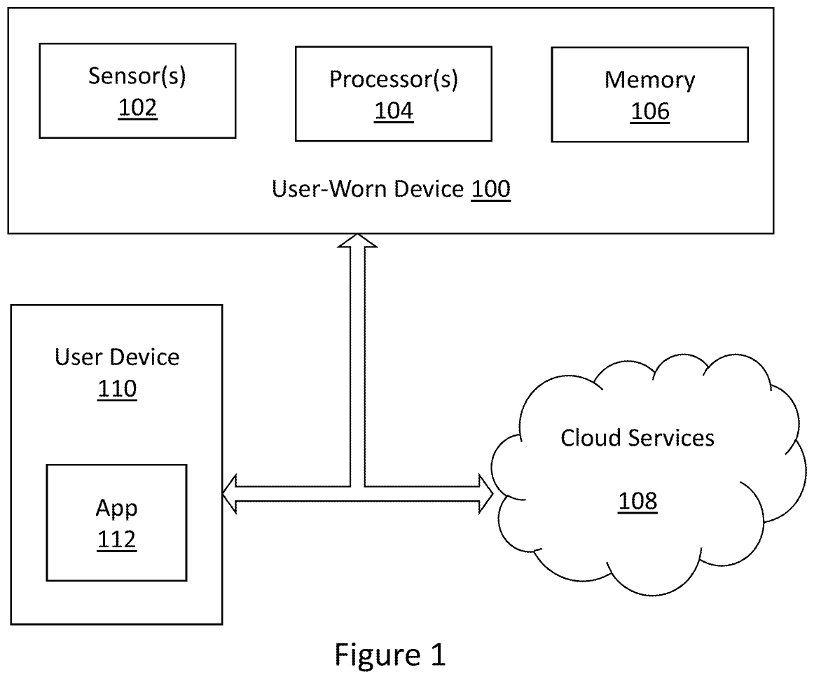

An example system according to the present disclosure is illustrated in FIG. 1. As seen therein, the system includes a user-worn device 100, having one or more physiological sensors 102, a processor 104, and memory 106. The user-worn device 100 is in communication with one or more cloud services 108 and a user interface device 110, on which an application 112 is executed that allows the user to interface with the user-worn device 100 and the cloud services 108. The user-worn device 100, cloud services 108, and user interface device 110 may be connected by an suitable means. For example, the user-worn device 100 and user interface device 110 may be connected via BLUETOOTH, WIFI, or like communication protocols. The user-worn device 100 and user interface device 110 may each be similarly connected to the cloud services 108 via a network such as the Internet.

According to one embodiment, the user-worn device 100 may be a hearing device having physiological sensors therein. For example, the hearing device may be a hearing aid, earbud, earphones, and the like. These devices may be in-the-ear, behind-the-ear, or over-the-ear hearing devices. Examples of such a hearing device are described in U.S. patent application Ser. No. 17/442,659 and WIPO Publication No. WO 2020/192956; in U.S. patent application Ser. No. 16/834,252 and U.S. Patent Application Publication No. 2021/0298670; in U.S. patent application Ser. No. 16/578,650 and U.S. Patent Application Publication No. 2021/0085254; and in U.S. patent application Ser. No. 16/481,152 and U.S. Patent Application Publication No. 2019/0394585, the entireties of which are incorporated herein by reference. Briefly, according to these examples, the hearing device includes a microphone, speaker, and processor. The hearing device further includes a light source and photodetector that may operate as a photoplethysmography (PPG) sensor, the PPG sensor corresponding to the at least one sensor 102 of the user-worn device 100. Light from the light source is emitted into tissue defining the user's ear canal, is reflected back, and detected by the photodetector. Signals representing the detected reflected light may be analyzed to determine various physiological conditions and/or vital signs, such as heart rate, oxygen saturation, and the like. Of course, other embodiments of the user-worn device 100 may be devices other than hearing devices, and/or may include other or additional sensors to the PPG sensor. For example, such devices may wrist worn and/or include sensors such as pedometers, accelerometers, and the like.

Data collected by the sensors 102 of the user-worn device 100 may be stored locally in the memory 106 of the user-worn device 100 for local processing and analysis of the data by the processor 104 of the user-worn device 100. Alternatively or additionally, the data may be transmitted to the cloud services 108 and/or the user interface device 110 for processing and/or storage. The data analysis may determine metrics such as vital signs, average values of vital signs over given time periods, a length of time of intense activity performed by the user, a number of steps, and the like.

The cloud services 108 may include remote storage and analysis of the data, for example, including statistical analysis of individual data, aggregation and analysis of collective data from many users within a relevant peer group (e.g., based on age, gender, height, weight, activity level, physiological metrics such as vital signs, and the like), artificial-intelligence analysis, and the like.

The user interface device 110 may be any user accessible computing device. For example, the user interface deice 110 may be a smartphone, tablet, laptop computer, or the like. As with the user-worn device 100 and the cloud services 108, the user interface device 110 may also be configured to store and process data from the sensors 102 of the user-worn device 100. Alternatively or additionally, the results of such analysis may be transferred from the user-worn device 102 or the cloud services 108 to the user interface device 110. In some embodiments, the user interface device 110 may collect all raw and/or processed data from the user-worn device 100, and then provide that data (or further processed data) to the cloud services 108. In this manner, the user-worn device 100 does not necessarily need a connection to the cloud services 108, and instead may only be connected to the user interface device 110. When executed by the user on the user interface device 110, app 112 provides (e.g., visually or audible) data from the sensors 102, results of analysis of the data, physical activity goals, and the like. This information provided to the user can be current and/or historical (e.g., showing progress). The app 112 also provides additional interfacing functionalities described in more detail below. In addition to providing information to the user, the app 112 may receive information from the user. For example, the user may indicate types of activities performed, (e.g., running or bicycling), personal information (e.g., height and weight).

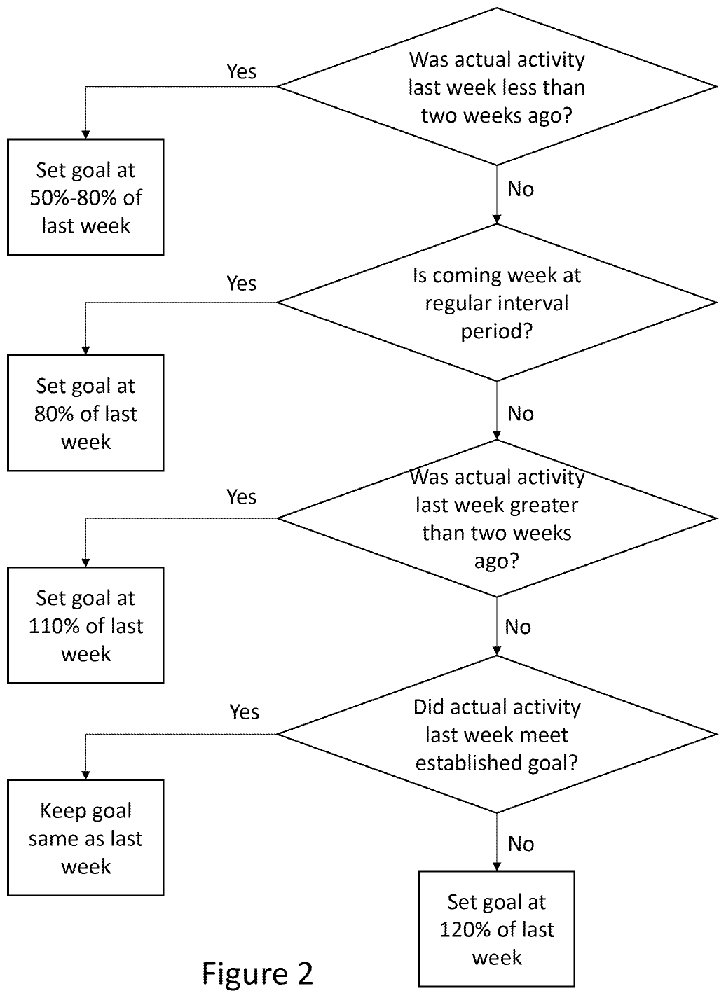

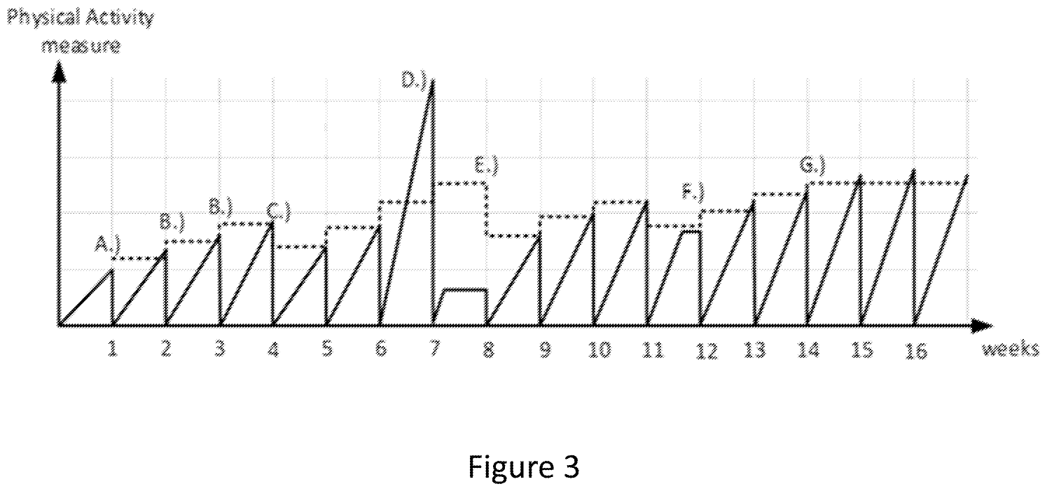

With reference to FIGS. 2 and 3, the following description relates to how goals may be determined by the app 112 and/or the cloud services 108. Briefly, the system (e.g., as described with respect to FIG. 1) may initially measure activity data for a given time period (e.g., one week) based on data from the sensors 102 of the user-worn device 100 and any information input by the user into the app 112 of the user interface device 110. From this, a current average amount and type of physical activity can be determined within that time period, and an initial goal may be set at or slightly above the initial activity level of the user. For activity beyond the initial time period, an actual activity level of the user may be determined based on the data from the sensors 102 and/or the analysis results of that data (e.g., identified metrics such as a number of steps, length of time of intense activity, vital signs, and the like).

According to the example flow chart for activity level goal setting in FIG. 2, the system sets an activity goal for a coming time period (e.g., a following week) based on various considerations. According to a first consideration, the activity goal is determined as a level within a predetermined percentage of a determined actual activity level of a prior time period (e.g., the preceding week). For example, the activity level goal for the coming time period may be an activity level that is greater than, and within 120% of, the activity level of the prior time period.

According to another consideration, when the actual activity level in the prior time period is greater than the actual activity level from a second prior time period (e.g., two weeks earlier and thus indicating an increase in actual activity level over two time periods), then the activity level goal for the coming time period may be increased relative to the prior time period goal by 0-20%. In one particular example, the activity level goal for the coming time period may only be 100-110% (a 0-10% increase) of the prior time period. In these cases, slowing the rate of increase of activity (by limiting the maximum activity level to be a 10% increase rather than a 20% increase) helps limit or avoid injuries because muscle strength increases faster than tendons, ligaments, bones, blood vessels, and the neurologic coordinative abilities to control the muscles. Further, unhealthy short term peak activities are not extra rewarded and translated into exaggerated goals; rather, the limited 10% increase represents an achievable amount. This can further help avoid demotivating an inactive user.

Similarly, if the actual activity level from the prior time period is lower than that from a second prior time period (e.g., two weeks earlier and thus indicating a decrease in actual activity level over two time periods not due to a lower goal in the prior time periods), the activity goal for the coming time period is limited. For example, the activity goal for the coming time period may be limited to between 50% and 80% (20%-50% lower) of the goal for the prior time period. Such a limit allows the user to sustain a reasonable amount when, for example, they were previously limited due to sickness, injury, vacation, or the like rather than a sudden lack of fitness.

For still another consideration, when an established guideline goal is reached, the activity goal for that physical activity measure is not further increased for the coming time period. For example, if the user achieves the World Health Organization guideline of 150 intensity minutes in a week, or 10,000 steps/day (e.g., as an average for a week), or the like in the prior time period, those goals are not further increased. That is, the goal is limited to the established guideline goal and not increased beyond 150 intensity minutes, 10,000 steps/day, or the like.

Additionally, the goal may be limited at regular intervals. For example, every fourth time period (e.g., every four weeks) the goal for the coming time period may be limited to a value less than the prior time period. Such a limit provides time for the user's body to adapt and recover. In one particular example, the goal for the coming time period is only 80% (20% lower) of the prior time period.

While FIG. 2 illustrate a linear decision path, it should be understood that the present disclosure is not so limited. Rather, any or all of the above considerations may be applied in other manners to determine goals. For example, the relevant information for each consideration may be input to a machine learning system, which is trained to output a goal activity level based on the input consideration information. Such a machine learning system may be embodied by the app 112 or the cloud services 108. In other embodiments, the goal may be determined as an average (or other statistical metric) of the goals suggested by each consideration. In some embodiments, the statistical metric may weight each consideration. In still other embodiments, the considerations may be evaluated in a different order than that shown in FIG. 2.

FIG. 3 illustrates an example of the above methodology for setting activity goals based on example determined actual activity levels. As seen in the figure, the actual activity level is identified by the solid line and the activity level goal is identified by the dashed line. The vertical axis of the graph represents a physical activity measure (e.g., steps, minutes of intense activity, and the like). The horizontal axis of the graph represents the time period (e.g., weeks).

During the first week of activity, no goal is set. Rather, an initial activity level of the user is determined. After the first week (as shown at point A), a first goal is set just beyond the initial week's activity of the user. Accordingly, the user is not necessarily given any goal well beyond their ability. For example, the user is not given a goal of immediately achieving 10,000 after being sedentary, and is thus not pushed to injury or demotivation due to failure. Rather, the initial goal is based on the user's current ability and behavior.

For each of the next two weeks (as shown at point B), the goal is increased by a predetermined amount. As noted above this amount may be 10% or 20%. In the example of FIG. 3, each increase is at 10%.

At week four (as shown at point C), the goal is lowered by 20% to allow the user's body to recover. This lowering is repeated every four weeks, which corresponds to the above-discussed regular interval.

During week six, it is seen that the user had an abnormally high activity level. Such an increase may be due to, for example, vacation, over-motivation, or the like. Nevertheless, the goal is only increased for week seven (as shown at point D) by a moderate amount, such as 10%.

Then in week seven, the user had an abnormally low activity level. Such a decrease may be due to, for example, illness, aches or injuries (e.g., from the previous week of high activity), or the like. In addition to detecting the decreased activity level, the system may also detect a heightened physiological stress level (e.g. through a decreased average heart rate variability, increased resting heart rate, or the like). As a result, the user's body may require significantly lower loads to properly recover. Therefore, the activity goal for the coming week eight is significantly lowered. However, the goal remains higher than the actual activity level of week seven.

In week eleven, despite having an easy ‘recovery’ week, the user's activity level still did not meet the goal. Therefore, the goal for week twelve (as shown at point F) is reduced and lower than it otherwise would be.

Finally, the goal for week fourteen (as seen at point G) corresponds to an established activity goal level (e.g., 150 minutes of intense activity or 10,000 steps). Despite the user achieving and even surpassing that goal in weeks thirteen through sixteen, the activity level goal is not increased beyond the established level.

The goal setting discussed may be directed to long term physical activities of at 12 weeks, 24 weeks, 48 weeks, or even longer. Thus providing rewards and motivations can help a user maintain interest in continuing to achieve their goals for the long term periods. Such rewards and motivations may be provided the app 112.

According to one example, the user may identify by input to the app 112 another person that may be informed of their actual activity and/or goals. The app 112 may transmit information related to the user's activity, physiological state, goals, historical data, and the like to the other person by email, SMS (or like) messaging, social media, and the like. The other person may then provide personal feedback to the user. Alternatively or additionally, the other person may respond via an interface with the app 112 so as to provide a message back to the user via the app 112. These messages may be predefined (e.g. “Great. Go on!”; “You had an easy week. Are you OK?”). In this manner, the other person serves as a source of encouragement and accountability for the user that is facilitated through the system, including the app 112. Similarly, the user may identify a health insurer, fitness institute, employer, or like third party to inform of their activity and goal achievements.

The app 112 may also facilitate motivation of the user financially. For example, the user may identify by input to the app 112 a definable amount of money and/or financial account information to and from which the amount of money may be transferred depending on whether the user achieves the goals. For example, the app 112 may facilitate payment of the amount of money from the user to a non-profit or non-governmental organization that supports a desirable cause for the user, to a family member, a favorable political party, or the like, to encourage achievement of the goals. However, if the user is unable to achieve the goal, the amount of many may instead be transferred to an opposing political party or organization with opposing views. Other financial incentives such as regular (e.g., weekly or monthly) premium payments, or cancellation fees, may also be required by the app 112 as motivation to continue physical activity and meet goals. Similarly, the app 112 may facilitate rewards to the user if their goals are met. For example, a user may receive discounts, refunds, coupons, gift cards, and the like via the app 112 in exchange for achieving their goals for a given time period.

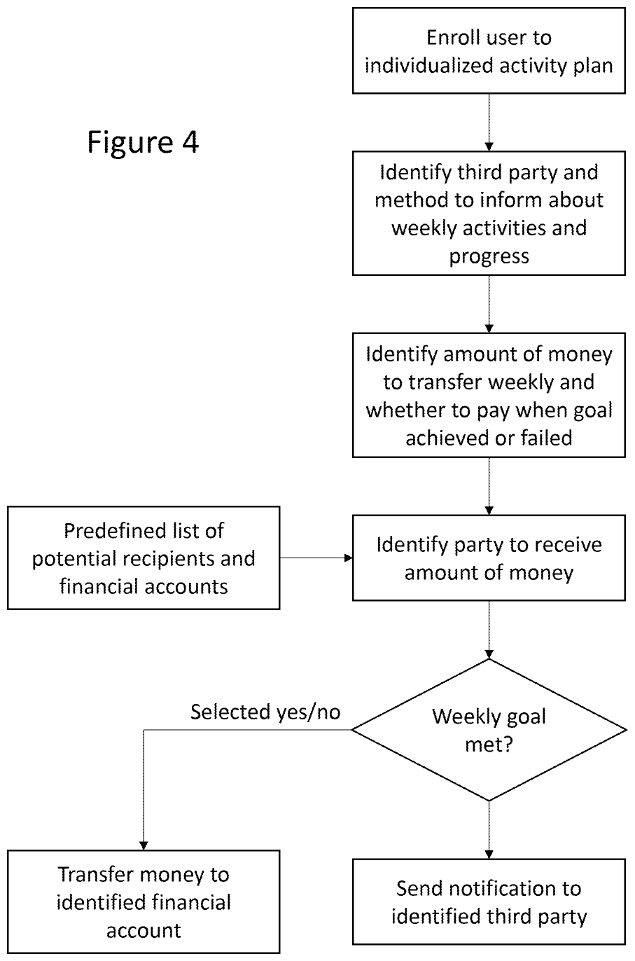

The above motivation and incentive features of the app 112 are further illustrated with respect to FIG. 4. According to the process therein, a user first enrolls in an individualized activity plan. Such a plan utilizes the user-worn device to track the user's activity level over predetermined time periods (e.g., a week) and determine activity level goals, as discussed above. As part of the incentive and motivation features of the app 112, the user then identifies a third party (e.g., a spouse or friend) to inform about their weekly progress, and a method of contacting that party (e.g., email, text messaging, social media). The user next identifies an amount of money, and whether it should be transferred if their goals for each time period are met, or if the user fails to meet the goals. As part of this indication, the amount of money (or multiples thereof) may be immediately withdrawn from the user's financial account by providing the user's financial account information to the app 112. Such withdrawn money may be managed by the distributor of the app 112, or like system manager. Alternatively, the amount of money may be set to withdraw and transfer automatically at the regular time period. Then a party is identified as a recipient of the money, including their financial account information so that the money can be fully transferred. This party may be another third party, such as the types of organizations discussed above. Alternatively, the user themselves may be the party, so that the money is refunded (or in effect, not withdrawn or paid out) if the user's goals are achieved, or so that the money can be used to give rewards (e.g., gift cards, coupons, and/or other goods and/or services). The app 112 may also be programmed with a predefined list of potential recipients for a user to choose from, and their financial account information (e.g., international bank account number (IBAN)) for such transfers. To facilitate financial management features of the app 112, the app 112 may be integrated with or otherwise include banking software.

Finally, each week the app determines whether the user's weekly goal(s) is met. Depending on the selected scenario for when to transfer the money (i.e., whether goal is met or not), the money is automatically transferred from the user's financial account or other account in which the money is held to the financial account for the identified recipient based on whether the user's goals are met. Regardless of whether the user meets the goals, the activity level and goal success information is sent to the identified third party that tracks weekly progress and activities. As noted above, the third party may return notifications to the user directly through the app 112. In addition, the app 112 may notify the user of a danger that the weekly goal may not be met. For the app 112 may notify a user that they have not meet the goal one day prior to the end of the week (or other time period).

While various features are presented above, it should be understood that the features may be used singly or in any combination thereof. Further, it should be understood that variations and modifications may occur to those skilled in the art to which the claimed examples pertain.

What is claimed is:

1. A system comprising:
a hearing device configured to be worn at an ear of a user, the hearing device comprising a microphone, a speaker, a processor, and at least one sensor configured to measure physiologic data or physical activity data of the user; and
a user computing device configured to:
obtain the physiologic data or physical activity data of the user from the hearing device,
for each of a plurality of recurring time periods, evaluate the obtained physiologic data or physical activity data for a current or prior time period and determine a current physical activity level based on the evaluated data for the current or prior time period, and adaptively determine a physical activity goal for a coming time period based on the determined current physical activity level, and
output the determined physical activity goal.

2. The system of claim 1, wherein the at least one sensor comprises a photoplethysmography sensor.

3. The system of claim 1, wherein the at least one sensor comprises an accelerometer.

4. The system of claim 1, wherein the recurring time period is one week.

5. The system of claim 1, wherein the determined physical activity goal for the coming time period is 50-80% of a physical activity goal of the prior time period when the current physical activity level is less than the determined physical activity level of two prior time periods earlier.

6. The system of claim 1, wherein the determined physical activity goal for the coming time period is less than, and at least 80% of, a physical activity goal of the prior time period when the coming time period is at a predetermined regular interval.

7. The system of claim 1, wherein the determined physical activity goal for the coming time period is greater than, and at most 110% of, a physical activity goal of the prior time period when the current physical activity level is greater than the determined physical activity level of two prior time periods earlier.

8. The system of claim 1, wherein the determined physical activity goal for the coming time period is the same as a physical activity goal for the prior time period when the current physical activity level meets or exceeds an established guideline activity goal.

9. The system of claim 1, wherein the determined physical activity goal for the coming time period is more than 100% and at most 110% of a physical activity goal for the prior time period when the current physical activity level meets or exceeds an established guideline activity goal.

10. The system of claim 1, wherein the determined physical activity goal for the coming time period is the same as a physical activity goal for the prior time period when the current physical activity level does not meet or exceed an established guideline activity goal.

11. The system of claim 1, wherein the determined physical activity goal for the coming time period is 110-120% of a physical activity goal for the prior time period when the current physical activity level does not meet or exceed an established guideline activity goal.

12. The system of claim 1, wherein the user computing device is further configured to:
transfer money from the user to a third party based on whether the determined current physical activity level meets or exceeds a physical activity goal for the prior time period.

13. The system of claim 1, wherein the user computing device is further configured to:
transfer money from the user to a third party based on whether the determined current physical activity level does not meet or exceed a physical activity goal for the prior time period.

14. The system of claim 1, wherein the user computing device is further configured to:
notify a third party of whether the determined current physical activity level meets or exceeds a physical activity goal for the prior time period.

15. The system of claim 1, wherein the user computing device is further configured to:
notify a third party of whether the determined current physical activity level does not meet or exceed a physical activity goal for the prior time period.

16. The system of claim 1, wherein the user computing device is further configured to:
notify the user when the current physical activity level does not meet or exceed a physical activity goal of a current time period, prior to expiration of the current time period.
